(19) United States
(12) Patent Application Publication
(10) Pub. No.: US 2015/0346174 A1
(43) Pub. Date: Dec. 3, 2015

(54) EXPOSURE MONITORING

(71) Applicant: INDUSTRIAL HYGIENE RESOURCES, LTD., Boise, ID (US)

(72) Inventors: Harry J. BEAULIEU, Boise, ID (US); Matthew C. CALL, Boise, ID (US)

(21) Appl. No.: 14/601,536

(22) Filed: Jan. 21, 2015

Related U.S. Application Data
(60) Provisional application No. 62/005,689, filed on May 30, 2014.

(51) Int. Cl. G01N 33/00; A62B 9/02; G01F 3/00; A62B 23/02; B01D 46/00; G01N 15/06; A62B 7/10; A62B 18/02

(52) U.S. Cl. CPC G01N 33/0036; A62B 7/10; A62B 9/02; A62B 18/02; A62B 23/025; B01D 46/0084; G01N 15/06; G01F 3/00

(57) ABSTRACT

A filter medium of an air purifying respirator captures one or more substances from the air in a worker's environment as the worked breathes. Air contaminants captured by the respirator filter media can be subsequently analyzed and measured. An air flow meter placed at an air channel of the respirator measures air flow and/or total air volume inspired by the worker wearing the respirator. Personal chemical exposure of the worker can be assessed from the filter media measurements and air flow data.

### CROSS-REFERENCE TO RELATED APPLICATIONS

This application claims the filing date benefit of U.S. Provisional Patent Application Ser. No. 62/005,689, filed on May 30, 2014, and titled “EXPOSURE MONITORING,” the entire contents of which are hereby incorporated by reference.

### BACKGROUND

1. Technical Field

The present disclosure relates generally to monitoring exposure to a person. In particular, the present disclosure relates to measuring, monitoring, and/or estimating air contaminant concentrations otherwise inspired by a person.

2. Description of Related Art

A current practice in the field of industrial hygiene typically involves measuring a worker's exposure to chemical air contaminants by placing an air sampling device in the personal breathing zone of the worker during a representative work shift. For example, a worker may have a shift that lasts 8-12 hours, during which time the air sampling device would be continuously worn. Current sampling technology and methodology for measuring air contaminants may typically employ an air sampling pump that uses a standardized and documented flow rate. Such a flow rate may incorporate generalized assumptions of the worker's breathing rates during the shift. Passive sampling badges and/or data-logging dosimeters (including, for example, electrochemical cell-based devices) are utilized for detecting some gases and vapors that may be present in the worker's environment.

In practice, actual worker breathing rates may vary greatly for each individual worker and may be affected by various factors, including worker body size, fitness level, work rate, exertion level, environmental conditions, and other factors. In addition, the establishment of occupational exposure limits of a chemical by the Occupational Safety and Health Administration (“OSHA”), i.e., OSHA permissible exposure limits (“PELs”), is typically based in part on toxicological data expressed as a dose of the chemical into the body of a test species, or worker, which dose is directly related to the breathing rate and ultimately the volume of air inspired by the worker. A worker performing hard, physical labor in hot conditions might be expected to breathe a significantly greater amount of air (and therefore chemical air contaminant) as opposed to a sedentary worker.

As an example, the permissible exposure limit (PEL) for inorganic lead (Pb) in air in a workplace in the U.S. is 50 micrograms of lead per cubic meter of air, averaged over an 8-hour work shift (i.e., the PEL for lead is 50 μg/m³·8 hour, as a time-weighted average). This regulatory exposure limit is intended to protect the health of the worker, knowing that with this exposure, he might be carrying lead in his body at a level as high as 30 micrograms of lead per deciliter of whole blood (30 μg/dL). However, measuring this exposure using current methods could result in an extremely inaccurate exposure measurement because of variations in work rates, and therefore respiration rates, between sedentary and active workers.

For example, published occupational limits for controlling exposures of workers to ozone (O₃) gas (CAS # 10028-15-6) addresses the level of work rate as a factor affecting the allowable limit. One recognized occupational exposure limit (“OEL”) for ozone gas becomes more stringent with increasing work rate in order to prevent adverse effects on pulmonary function of the worker during a work shift. For light work, the allowable limit is 0.10 ppm for 8 hours; for moderate work, the limit is 0.08 ppm; and for heavy work, the limit is 0.05 ppm. For work periods of less than two hours, the allowable limit is 0.2 ppm at any work rate. Exposure rates for various substances are likewise expressed in terms of work rates. However, assessing which work rate applies in any given circumstance may be subjective, inconsistent, and/or difficult to clearly define.

### SUMMARY

In one embodiment a system for monitoring an environment is disclosed. The system includes an air flow channel, an air flow sensor in fluid communication with the air flow channel, a memory adapted to receive air flow data generated by the air flow sensor, and a filter medium in fluid communication with the air flow channel.

In another embodiment, a method for monitoring an environment is disclosed. The method includes collecting particles of a substance in a filter medium, measuring an air flow rate passing through the filter medium, calculating a cumulative air flow, measuring a mass of the particles in the filter medium, and calculating an amount of the substance in the environment based on the mass of the particles and the cumulative air respired.

The present disclosure will now be described more fully with reference to the accompanying drawings, which are intended to be read in conjunction with both this summary, the detailed description, and any preferred or particular embodiments specifically discussed or otherwise disclosed. This disclosure may, however, be embodied in many different forms and should not be construed as limited to the embodiments set forth herein; rather, these embodiments are provided by way of illustration only so that this disclosure will be thorough, and fully convey the full scope thereof to those skilled in the art.

### BRIEF DESCRIPTION OF THE DRAWINGS

Non-limiting and non-exhaustive embodiments of the present disclosure are described with reference to the following figures, wherein like reference numerals refer to like parts throughout the various views unless otherwise specified.

FIG. 1 illustrates an exposure monitoring device according to embodiments of the present disclosure;

FIG. 2 is a perspective view cutaway drawing of an exposure monitoring device according to embodiments of the present disclosure;

FIG. 3 is an illustration of use of an exposure monitoring device according to an embodiment of the present disclosure;

FIG. 4 is side view cutaway drawing of an exposure monitoring device according to embodiments of the present disclosure; and

FIG. 5 is an illustration of an embodiment of an exposure monitoring device having an air flow meter integrated into the filter cartridge.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present disclosure. Also, common but well-understood elements that are useful or necessary in a commercially feasible embodiment are often not depicted in order to facilitate a less obstructed view of these various embodiments of the present disclosure.

### DETAILED DESCRIPTION

In the following description, reference is made to exemplary embodiments in which the disclosure may be practiced. These embodiments are described in sufficient detail to enable those skilled in the art to practice the concepts disclosed herein, and it is to be understood that modifications to the various disclosed embodiments may be made, and other embodiments may be utilized, without departing from the spirit and scope of the present disclosure. The following detailed description is, therefore, not to be taken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the particular features, structures, or characteristics may be combined in any suitable combinations and/or sub-combinations in one or more embodiments or examples.

Embodiments of the present disclosure provide methods, apparatus, components, and/or techniques for measuring, monitoring, and/or estimating an amount of air inspired by a worker and/or analyzing concentrations of airborne matter in the air inspired by the worker or otherwise found in the environment around the worker. According to various embodiments of the present disclosure, an air flow sensor attached to a conventional respirator worn by a worker can measure an amount of air inspired by the worker while she wears the respirator. In some embodiments, the total volume and/or mass of inspired air may be measured, calculated, and/or stored.

According to various embodiments of the present disclosure, the amount of matter that has been captured by filter media in the respirator worn by the worker may be measured and/or analyzed. In embodiments, such matter may comprise air contaminants, including potentially hazardous material. In other embodiments, such matter comprises other substances of interest.

Embodiments of the present disclosure comprise carrying out analytical methods to determine the amount of matter in the filter media at the end of a work shift (i.e., matter that could otherwise have been inspired by the worker). Considering the volume of air inspired by the worker while wearing the respirator and the amount of matter filtered by the respirator, one may accurately estimate the personal exposure to matter in the air at the location where the respirator was worn. The results of such calculations can be correlated to standard exposure monitoring methods to estimate the amount of certain substances in the environment and to assess exposure of the worker to said substances.

In one particular illustrative example, a respirator is worn by a worker during a work shift. In this illustrative example, the respirator includes an air flow sensor that measures the amount of air passing through a filter media cartridge on the respirator and thus reflects the air inspired by the worker. In this particular illustrative example, the cumulative total volume and/or mass of air inspired by the worker during the shift may be determined by accessing the data gathered by the air flow sensor and adding together the volume and/or mass of each breath.

In this illustrative example, the concentration of selected contaminants in the air where the worker carried out the work shift can be calculated from the amount of that particular contaminant in the filter media and the total amount air inspired by the worker during the shift. The amount of a selected contaminant can be measured in the filter media using techniques that are now known or that may be known in the future. As would be understood by one of ordinary skill in the art having the benefit of this disclosure, the analysis completed in the foregoing illustrative example method may result in a higher degree of accuracy for chemical exposure monitoring for an individual in the workplace compared to traditional methods. In particular, the resulting data may be a more accurate estimation of exposure and dose of the contaminant to the particular worker because the worker's actual breathing rate is incorporated into the analysis rather than using generalized assumptions about the worker's breathing rate and total amount of air inspired. Additionally, approaches set forth in the present disclosure may present the advantage of determining contaminant air concentrations without the worker wearing an air sampling pump and separate sampling media or other bulky equipment.

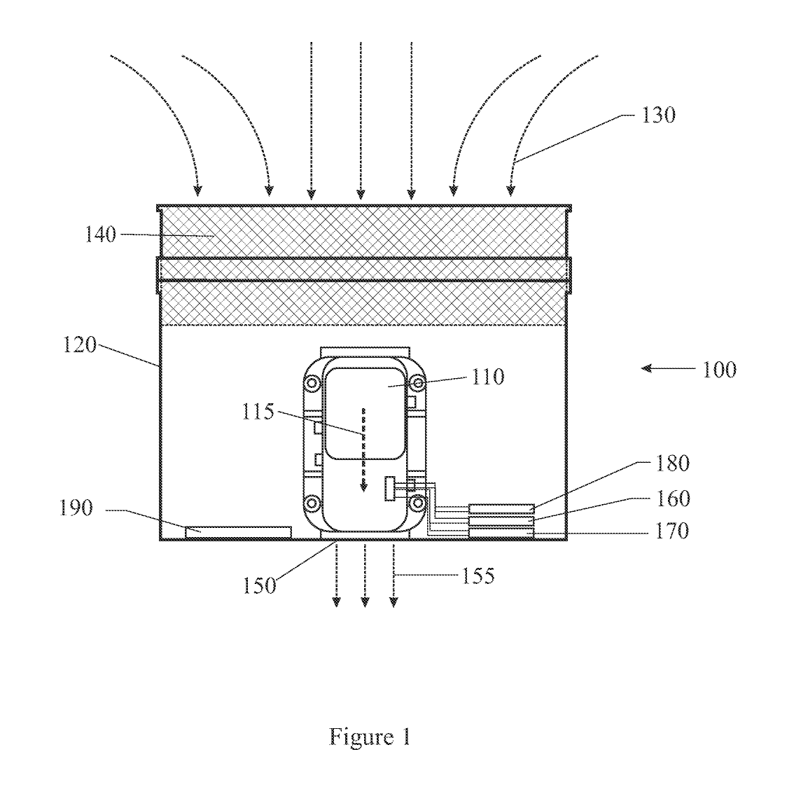

Referring now to FIG. 1, an exposure monitoring device 100 according to embodiments of the present disclosure is depicted. FIG. 1 illustrates an air flow sensor 110 inside a respirator adapter housing 120. According to various embodiments, the respirator adapter housing 120 may comprise an assembly that can attach to a respirator mask. In one embodiment, housing 120 can attach to a respirator mask at the air flow inlet, so that air passes through flow sensor 110 before flowing into the respirator mask. In another embodiment, housing 120 attaches to an air flow outlet, so that exhaled air may then pass from the respirator, into housing 120, and through air flow sensor 110.

According to embodiments, housing 120 comprises an inlet for air flow 130, where air may pass through the filter media 140 before reach an internal chamber of the assembly where air flow sensor 110 is positioned to receive essentially all air flow passing through housing 120. As depicted in FIG. 1, according to embodiments, of the assembly, air flow sensor 110 may be placed in an internal chamber of housing 120 in the path of air exiting the filter media 140.

According to embodiments, air flow exiting airflow sensor 110 is directed through the assembly outlet 150 to an attached respirator (not depicted in FIG. 1). Airflow 155 exiting housing 120 may be directed to a respirator mask. According to embodiments, various types of air flow meters may be utilized to measure air speed, air flow, and/or air volume of air passing through. In some embodiments, various types of heat-transfer-based air flow sensors are utilized. Such sensors may include a “hot wire” type air flow meter. In other embodiments, a vane-based or differential pressure-based air flow sensor is utilized. In other embodiments, a microelectromechanical systems (“MEMS”) based air flow sensor is utilized.

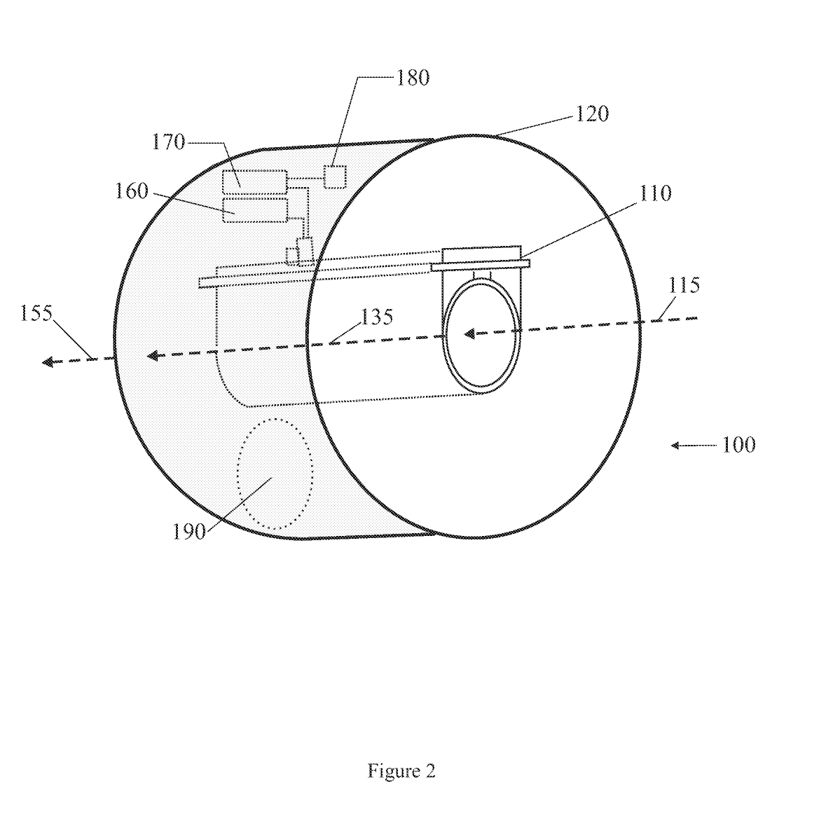

Referring now to FIG. 2, a cross-sectional view of an embodiment of a filter cartridge adapter is depicted. In embodiments, a microcontroller 160, storage memory 170, communications port 180, and battery 190 may be placed within the respirator adapter housing 120. According to various embodiments, the microcontroller 160 is programmed to drive the air flow sensor 110 and/or receive data generated by the air flow sensor 110. The memory 170 can store data generated by the air flow sensor 110 and allow for later data retrieval. The communications port 180 can provide for transmitting data generated by the air flow sensor 110. Such data transmission may be carried out by wireless protocols, by wired protocols, and/or by other known signal transmission means as discussed herein, and/or as known in the art at the time of filing, and/or as developed after the time of filing. In one embodiment, data generated by the air flow sensor 110 may be transmitted in addition to or instead of storing such data at a memory storage device 170.

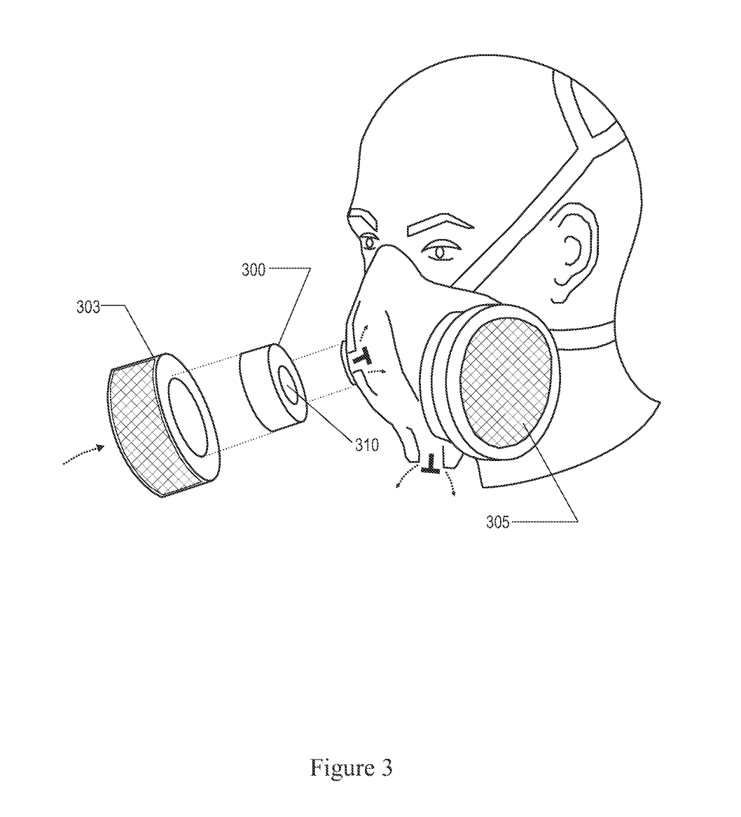

Referring now to FIG. 3, an embodiment of a filter cartridge adapter 300 having an air flow sensor 310 is illustrated. As shown, a removable filter cartridge 340 attaches to the filter cartridge adapter 300, which in turn attaches to the respirator 350 at the respirator inhalation valve 360. In this manner, air that is inspired by the worker passes first through the filter media 340, then through the air flow sensor 310, then through the respirator inhalation valve 360 into the face piece of the respirator 350. In embodiments, an air flow sensor is placed within or at both filter cartridges 303, 305.

In embodiments, the respirator inhalation valve and exhalation valve comprise one-way valves to direct air flow in from the filter media and air flow meter to the respirator, and out the exhalation valve. Thus, the air flow sensor can measure air flow that has passed through the filter cartridge but not yet into the respirator. In other words, in various embodiments, the air flow sensor measures air flow in only one direction. In other embodiments, an air flow meter is placed at the respirator exhalation value and is adapted to measure air that has been exhaled from the worker.

In embodiments, the filter cartridge adapter comprises threaded attachment mechanisms to attach to the filter cartridge and to the respirator. Such threaded attachment mechanisms are adapted to fit standard threading configurations commonly found on respirator masks. In other embodiments, the filter cartridge adapter can attach to the filter cartridge and/or to the respirator by other attachment mechanisms as discussed herein, and/or as known in the art at the time of filing, and/or as developed after the time of filing.

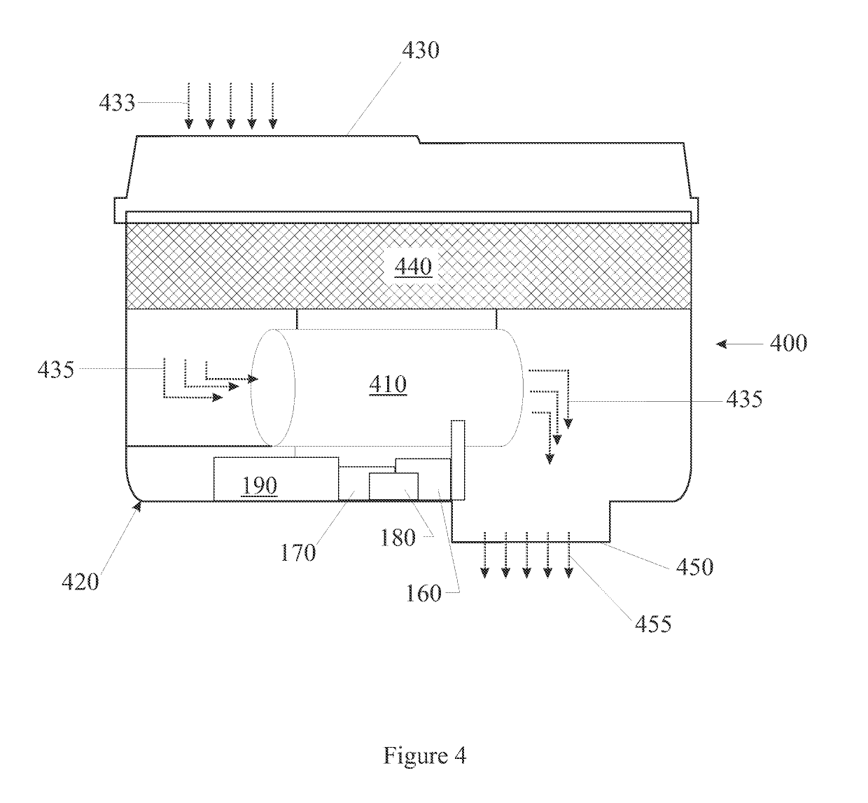

FIG. 1 depicts an air flow sensor 110 having an air flow direction 115 that is parallel to, and in-line with, the general flow direction from the filter media 140 to the respirator. In other embodiments, the air flow sensor 110 is placed within the respirator adapter housing 120 at various orientations. Referring now to FIG. 4, in one embodiment of an exposure monitoring device 400, the air flow sensor 410 is oriented so that air flow through it is essentially perpendicular to the general flow direction from the filter media 440 to the respirator. Airflow 433 can enter filter media 440 via inlet 430. An air flow channel within the respirator adapter housing 420 may direct air flow 435 to pass from inlet 430 through air flow sensor 410. Exiting airflow 455 may be directed to a respirator inhalation valve via the respirator outlet 450.

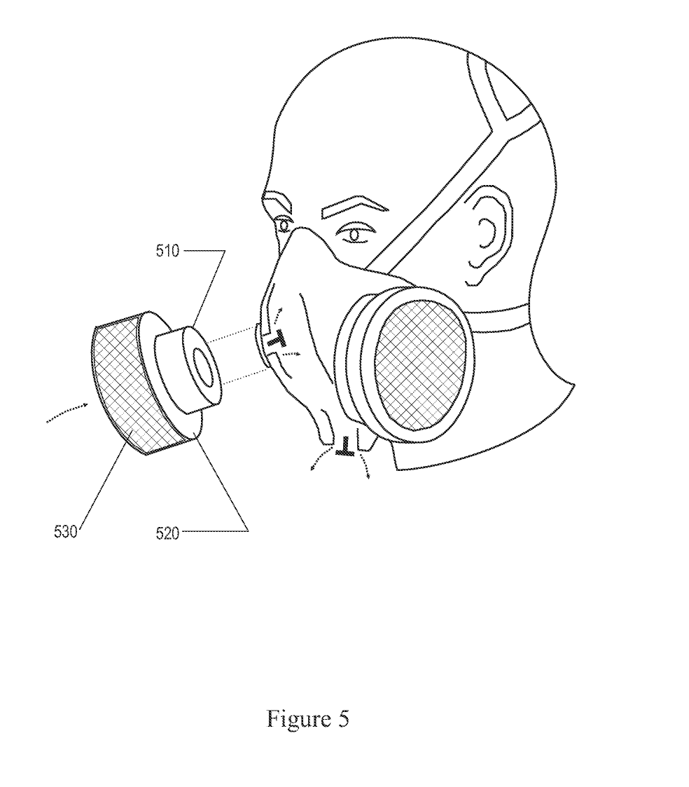

Referring now to FIG. 5, in various embodiments, an air flow sensor 510 may be embedded within a filter cartridge 520. In one embodiment, an air flow sensor 510 is built into a disposable filter cartridge 520. The air flow sensor 510 may be removed from the cartridge 520 after completion of a work shift before filter media 530 is analyzed for contaminants. The air flow sensor can be re-used after downloading data stored therein.

In operation, according to various embodiments of the present disclosure, one or more air flow sensors are installed within or attached to an air respirator and worn by a worker. As the worker progresses through the work shift, each breath is measured by the air flow sensor and logged in a memory. As each breath is made by the worker, the air flow sensor measures the mass and/or the volume of air passing therethrough. Additional relevant data may be measured and recorded including, but not limited to, the ambient temperature, air pressure and other conditions of the worker's environment. In one embodiment, data is transmitted to an on-board memory within the respirator adapter housing. In another embodiment, data is transmitted wirelessly to a data receiving apparatus in the vicinity of the worker.

Each time the worker inhales, air in the environment of the worker is drawn into the filter cartridge, through the filter media, through the air flow meter, and into the respirator mask. Vapors, particles, and/or other matter in the air may be collected by the filter media and remain embedded there. Throughout the course of the work shift, such substances may accumulate in the filter media.

After the worker completes a work shift, the filter cartridge(s) are collected for analysis. Data from the flow sensor(s) regarding air flow rate and/or volume are retrieved. The cartridges may then be sent to an industrial hygiene laboratory or like location where the filter media within the cartridges are analyzed for the substances, including chemical air contaminants, to which the worker was exposed. The laboratory may use standard and accepted methods of analyses of the chemical air contaminants, e.g., NIOSH method for inorganic lead (Pb). As would be understood by one of ordinary skill in the art having the benefit of this disclosure, the laboratory may use more volume of reagent and larger glassware to digest cartridge media, as compared to standard methods of handling conventional air samples (i.e., a filter cartridge having a relatively large filter media may call for more reagent than a relatively small cartridge with membrane filters).

According to embodiments, analyses of filter media may result in a determination of the amount of various substances, including chemical air contaminants, in the filter media. Such measurements may be made with respect to the volume, mass, molar quantity, or other forms of measurement of substance. It may be assumed that had the worker not been wearing the respirator mask, he would have breathed an near equivalent amount of substance into his lungs, given his breathing rate, level of exertion, and/or work rate. This amount of contaminant, e.g., inorganic lead (Pb), may be a more accurate estimation of the personal dose to which that worker was exposed, as compared to current methods of exposure monitoring that do not consider variations in breathing rates, work rates, and exertion levels for individual workers. With a more accurate estimation of dose of the chemical into the body of the worker, this information could be more aligned with basic toxicological data for that chemical.

According to various embodiments, with data of the total air inspired during a work shift and total amount of chemical contaminants in the filter, an inference of the amount of contaminants in the work environment may be made. For example, but not to be taken in a limiting sense, the total mass of a substance that was measured in the filter media may be divided by the total volume of air inspired in order to more accurately measure the exposure to a worker and more closely estimate the dose received by a person in the work environment. In this example, the measured exposure represents the time period that the respirator was worn, but could also be extrapolated to represent longer periods of time. The work shift exposure may be compared to an applicable PEL.

Embodiments of the present disclosure may be practiced in the field in remote locations by merely distributing the filter cartridges, air flow sensors, and respirators to workers, providing instructions for their use of same during work shifts, and then collecting the used filter cartridges after completion of their work shifts. The used cartridges and air flow memory units may then be transported to an appropriate laboratory for analyses. The laboratory may download data from the air flow rate sensor, remove the media from the respirator cartridges, digest (or desorb) the media, and analyze digestate (or extract) with a standard methodology, e.g., to determine total amount of a substance (e.g., lead) on the filter media. The laboratory may report the results of these analyses to an industrial hygienist or other technician. Calculations may be performed to determine the concentration of the contaminant in air (e.g., as mg/m³, parts per million, as a concentration percentage, or in other forms including but not limited to a ratio representing the amount of substance in the environment) over the period during which the respirator was worn. In alternative embodiments, results of such analyses could be transmitted to any person and/or entity that may have interest in the results. The industrial hygienist or other recipient could then have personal exposure data for the worker to that chemical air contaminant, with the particular worker's work rate incorporated into the evaluation.

According to other embodiments, aspects of the present disclosure could be automated to varying extents. In one example, data from the air flow meter(s) could be uploaded automatically to a database and incorporated into worker exposure calculations following analysis of the corresponding filter cartridge.

Although the present disclosure is described in terms of certain preferred embodiments, other embodiments will be apparent to those of ordinary skill in the art, given the benefit of this disclosure, including embodiments that do not provide all of the benefits and features set forth herein, which are also within the scope of this disclosure. It is to be understood that other embodiments may be utilized, without departing from the spirit and scope of the present disclosure.

What is claimed is:

1. A system for monitoring an environment, comprising:
an air flow channel;
an air flow sensor in fluid communication with the air flow channel;
a memory adapted to receive air flow data generated by the air flow sensor; and
a filter medium in fluid communication with the air flow channel.

2. The system of claim 1, further comprising a one-way valve in fluid communication with the air flow channel.

3. The system of claim 1, further comprising a microcontroller in electrical communication with the air flow sensor.

4. The system of claim 1, further comprising a power source.

5. The system of claim 4, wherein the power source comprises a battery.

6. The system of claim 1, further comprising a filter cartridge adapter comprising a housing for the air flow sensor, the memory, and at least a portion of the air flow channel.

7. The system of claim 6, wherein the filter cartridge adapter is removably attachable to a respirator mask.

8. A method for monitoring an environment, comprising:
collecting a substance in a filter medium;
measuring an air flow rate passing through the filter medium;
calculating a cumulative air flow;
measuring an amount of the substance in the filter medium; and
calculating a ratio of the substance in the environment based on the amount of the substance and the cumulative air respired.

9. The method of claim 8, wherein collecting the substance in the filter medium further comprises collecting the substance in the filter medium within a cartridge that is attached to a respirator mask.

10. The method of claim 8, wherein calculating the ratio of the substance in the environment further comprises calculating a volumetric percentage of the substance in the environment.

11. The method of claim 8, wherein calculating the ratio of the substance in the environment further comprises calculating a mass percentage of the substance in the environment.

12. The method of claim 8, wherein measuring the amount of the substance in the filter medium further comprises:
desorbing the substance in a solvent and
measuring a mass of the substance.

13. An apparatus for monitoring an environment of a worker, comprising:
an air flow sensor in fluid communication with a respirator worn by the worker and
a filter medium in fluid communication with the air flow sensor.

14. The apparatus of claim 13, further comprising a memory adapted to receive air flow data generated by the air flow sensor.

15. The apparatus of claim 13, further comprising a one-way valve in fluid communication with the air flow sensor.

16. The apparatus of claim 13, further comprising a microcontroller in electrical communication with the air flow sensor.

17. The apparatus of claim 13, further comprising a power source.

18. The apparatus of claim 17, wherein the power source comprises a battery.

19. The apparatus of claim 13, further comprising a filter cartridge adapter comprising a housing for the air flow sensor and the filter medium.

20. The system of claim 19, wherein the filter cartridge adapter is removably attachable to the respirator mask.
